(19) United States
(12) Patent Application Publication
(10) Pub. No.: US 2009/0291067 A1
(43) Pub. Date: Nov. 26, 2009

(54) Composition for treating cancer comprising adult stem cell culture or its fraction

(75) Inventors: Byung-Soon Park, Seoul (KR); Won-Serk Kim, Seoul (KR); Jong-Hyuk Sung, Seongnam-si (KR)

Correspondence Address: THE NATH LAW GROUP, 112 South West Street, Alexandria, VA, 22314, US

(73) Assignee: PROSTEMICS CO., LTD., Seoul (KR)

(21) Appl. No.: 12/453,539

(22) Filed: May 14, 2009

(30) Foreign Application Priority Data
May 22, 2008 (KR) 10-2008-0047498

(51) Int. Cl. A61K 35/12

(52) U.S. Cl. 424/93.7

(57) ABSTRACT

The present invention provides a pharmaceutical composition for treating cancer, comprising a culture of adult stem cells or a fraction of the culture as an active ingredient. The culture of adult stem cells and its fraction, especially a specific fraction of adult stem cell culture, inhibit proliferation of a variety of cancer such as melanoma, pancreatic cancer, breast cancer, hepatic cancer, gastric cancer, colon cancer, lung cancer, and cervical cancer, thereby having excellent cancer-treating activity. The composition according to the present invention includes, not stem cells, but a complex of active proteins secreted from the stem cells, and thus both pharmaceutical problems in formulation and individual variation, which usually occurred when using stem cells, can be minimized. And also, side effects caused by direct administration of cells into the human bodies can be thoroughly prevented.

### CROSS-REFERENCE TO RELATED PATENT APPLICATIONS

This application claims the benefit of Korean Patent Application No. 10-2008-0047498, filed on 22 May, 2008, in the Korean Intellectual Property Office, the disclosure of which is incorporated herein in its entirety by reference.

### BACKGROUND OF THE INVENTION

1. Field of the Invention

The present invention relates to a pharmaceutical composition for treating cancer, comprising a culture of adult stem cells or its fraction as an active ingredient.

2. Description of the Related Art

Lots of patients have been suffered from a variety of cancer such as gastric cancer, lung cancer, hepatic cancer, colon cancer, breast cancer, skin cancer (e.g., melanoma), etc. In order to treat cancer, various chemically synthesized anti-cancer agents, such as gemcitabine, are being used or radiation therapy is being performed. Recently, molecular-biological agents against carcinogenesis have been developed. For example, there have been developed bevacizumab selectively binding to a vascular endothelial growth factor (VEFG); cetuximab as a monoclonal antibody targeting an epidermal growth factor receptor (EGFR); erlotinib and gefitinib as EGFR-activating tyrosine kinase; and tipifarnib as an inhibitor of K-ras and farnesyl transferase.

Meanwhile, Korean Laid-Open Patent Publication No. 10-2007-0036289 discloses a composition for treating cancer, such as breast cancer, hepatic cancer, and pancreatic cancer, comprising mesenchymal stem cells (MSCs) expressing a suicidal gene. The suicidal gene is a gene capable of converting a non-toxic prodrug to corresponding cytotoxic anti-cancer drug. For example, cytosine deaminase has an activity of converting 5-fluorocytosine (5-FC) to cytotoxic anti-cancer agent, 5-fluorouracil (5-FU).

However, when stem cells, e.g., MSCs, are intended to be directly administered for cancer treatment, it is difficult to reproductively formulate stem cells while maintaining their viability at desired level. Furthermore, since clinical application using stem cells may show high individual variation according to their sources, it is difficult to obtain uniform therapeutical effects. In addition, direct administration of stem cells per se cannot exclude the possibility of occurring newly-generated side effects, e.g., tumor formation.

### SUMMARY OF THE INVENTION

The present inventors have carried out extensive research in order to improve problems of the prior arts and develop a method for treating a variety of cancer such as melanoma, pancreatic cancer, breast cancer, hepatic cancer, gastric cancer, colon cancer, lung cancer, and cervical cancer. As a result, surprisingly, we found that a culture of adult stem cells, especially its specific fractions, have high anti-cancer activity. In particular, since it is possible to avoid direct administration of cells to human bodies, side effects caused by direct administration of cells into the human bodies can be thoroughly prevented.

In accordance with an aspect of the present invention, there is provided a pharmaceutical composition for treating cancer, comprising a culture of adult stem cells or a fraction of the culture as an active ingredient.

The culture of adult stem cells may be a supernatant obtained through centrifuging a culture solution of the adult stem cells; a concentrated product of the supernatant; or a lyophilized product of the supernatant.

The fraction of the adult stem cell culture may be a fraction obtained by fractionating a supernatant obtained through centrifuging a culture solution of the adult stem cells such that proteins having 36 to 700 kDa, preferably 36 to 45 kDa or 95 to 200 kDa, more preferably 36 to 45 kDa or 130 to 200 kDa, of molecular weight are contained in the resultant fraction; a concentrated product of the fraction; or a lyophilized product of the fraction.

The culture solution of the adult stem cells may be obtained by sub-culturing the adult stem cells in a serum-containing medium, followed by culturing the sub-cultured adult stem cells in a serum-free medium. The serum-containing medium is a Dulbecco's Modified Eagle's Medium (DMEM) supplemented with 1 to 2 mM of glutamine, 0.5 to 1 mM of sodium pyruvate, 0.1 to 10% of fetal bovine serum (FBS), 1% of antibiotics (100 IU/ml), and 1 to 4.5 g/L glucose. And, the serum-free medium may be a mixed medium of DMEM and Ham's F-12.

The adult stem cells may be derived from fat, bone marrow, umbilical cord blood, or placenta. And the cancer includes melanoma, pancreatic cancer, breast cancer, hepatic cancer, gastric cancer, colon cancer, lung cancer, or cervical cancer.

### BRIEF DESCRIPTION OF THE DRAWINGS

The above and other features and advantages of the present invention will become more apparent by describing in detail exemplary embodiments thereof with reference to the attached drawings in which:

FIG. 1 shows proliferation rates of rodent melanoma cells treated with an adipose stem cell culture solution;

FIG. 2 shows proliferation rates of human melanoma cells treated with an adipose stem cell culture solution;

FIG. 3 shows images of pancreatic cancer cells treated with an adipose stem cell culture solution, obtained using a microscope;

FIG. 4 shows images of pancreatic cancer cells treated with an adipose stem cell culture solution in various concentrations, obtained using a microscope;

FIG. 5 shows viability of pancreatic cancer cells treated with an adipose stem cell culture solution in various concentrations;

FIG. 6 shows proliferation rates of human breast cancer cells treated with an adipose stem cell culture solution;

FIG. 7 shows proliferation rates of human hepatic cancer cells treated with an adipose stem cell culture solution;

FIG. 8 shows proliferation rates of human gastric cancer cells treated with an adipose stem cell culture solution;

FIG. 9 shows proliferation rates of human colon cancer cells treated with an adipose stem cell culture solution;

FIG. 10 shows proliferation rats of human lung cancer cells treated with an adipose stem cell culture solution;

FIG. 11 shows results of in vivo inhibition test of melanoma growth, using an adipose stem cell culture solution;

FIG. 12 shows proliferation rates of melanoma cells treated with an umbilical cord blood stem cell culture solution and a bone marrow stem cell culture solution;

FIG. 13 shows proliferation rates of rodent melanoma cells treated with fractions of an adipose stem cell culture solution;

FIG. 14 shows proliferation rates of rodent cervical cancer cells treated with fractions of an adipose stem cell culture solution;

### DETAILED DESCRIPTION OF THE INVENTION

The present invention will now be described more fully with reference to the accompanying drawings, in which exemplary embodiments of the invention are shown.

It is found that a culture of adult stem cells and a fraction thereof, especially a specific fraction of adult stem cell culture, inhibit proliferation of a variety of cancer such as melanoma, pancreatic cancer, breast cancer, hepatic cancer, gastric cancer, colon cancer, lung cancer, and cervical cancer, thereby having excellent cancer-treating activity. In addition, the composition according to the present invention includes, not stem cells, but a complex of active proteins secreted from the stem cells, and thus both pharmaceutical problems in formulation and individual variation, which usually occurred when using stem cells, can be minimized. And also, side effects caused by direct administration of cells into the human bodies can be thoroughly prevented.

The present invention provides a pharmaceutical composition for treating cancer, comprising a culture of adult stem cells or a fraction of the culture as an active ingredient

The adult stem cells refer to multi-potent undifferentiated cells derived from a mammal including a human being, specifically from a human being. For example, the adult stem cells can be derived from adult cells, such as bone marrow, blood, brain, skin, adipocytes (that is, adipose tissues or adipose cells), umbilical cord blood, or Wharton's jelly of the umbilical cord. In the present specification, the “adult stem cells” include mesenchymal stem cells derived from adult cells.

Among the adult stem cells, adipose stem cells can be obtained from the adipose tissues discarded in a conventional liposuction process. Therefore, adipose stem cells, which can be obtained without invasive procedure, are preferably used in the present invention. The adipose stem cells may be obtained from adipose tissues or adipose cells of a mammal including a human being, specifically a human being, through procedures of liposuction, precipitation, treatment with an enzyme such as collagenase, and removal of a supernatant cells such as red blood cells via centrifugation, according to known methods, e.g., WO2000/53795 and WO2005/042730. Examples of the adipose tissues include brown or white tissues derived from subcutaneous tissue, omentum, viscera, breast gonad, or other adipose tissues, which can be easily obtained by using a conventional liposuction technique.

Adult stem cells can be cultured using a medium for culturing stem cells according to a conventional method. That is, stem cells derived from adult cells, such as bone marrow, blood, umbilical cord blood, and adipocytes can be cultured using conventional medium and culture condition that are suitable for culturing respective stem cells. Preferably, adult stem cells (e.g., adipose stem cells) may be sub-cultured in a serum-containing medium, and then cultured in a serum-free medium, thereby increasing the amount of proteins in the obtained culture or its fraction.

The serum-containing medium is a medium suitable for keeping and storing cell types identical to adipose stem cells, for example, a serum-containing Dulbecco's Modified Eagle's Medium (DMEM) or a serum-containing freeze-dried DMEM supplemented with 7 to 10 wt % of dimethyl sulfoxide (DMSO). The serum may be fetal bovine serum (FBS) and the amount thereof may be about 10 wt % based on the total amount of the serum-containing medium. When required, the medium may further include antibiotics, antifungal agents, and micoplasma inhibitors. The antibiotics may be conventional antibiotics used in culturing cells, such as penicillin and streptomycin. The antifungal agents include amphotericin B and the micoplasma inhibitors include tylosin, gentamicin, ciprofloxacin, or azithromycin. When required, the serum-containing medium may further include nutrients such as, glutamine and sodium pyruvate.

Preferably, the serum-containing medium may be a DMEM supplemented with 1-2 mM of glutamine, 0.5-1 mM of sodium pyruvate, 0.1-10% of FBS, 1% of antibiotics (100 IU/ml), and 1-4.5 g/L glucose. Culturing in the serum-containing medium may be performed, in a 5-10% CO₂ incubator, under the conditions of 90-95% of humidity and 35-39° C. of temperature. When required, a carbon source, such as sodium bicarbonate, may be added to the medium such that the final concentration thereof is in a range of 0.17-0.22 wt % and trypsin-EDTA may be added to the medium in order to promote cell growth. A cumulative doubling time may be maintained until cells being cultured in a flask reach 75-85% of confluence. For example, cells may be collected at 80% of confluence and then subsequent subculture may be performed.

Culturing in a serum-free medium may be performed using cell pellets obtained from the above subculture. The cell pellets may be obtained as follows: the culture solution is removed from the serum-containing medium, washed with a phosphate buffer and then treated with trypsin-EDTA. The resultant cell suspension is centrifuged to obtain cell pellets, followed by washing with a phosphate buffer two or three times.

The serum-free medium may be a medium supplemented with one or more nutrients, specifically a 1:1 mixture of DMEM and Ham's F-12 (see SIGMA, Cancer Research Vol 47, Issue 1 275-280) supplemented with nutrients such as L-glutamine, sodium pyruvate, and sodium bicarbonate. When the mixed medium is used, the growth and homeostasis of stem cells may be effectively maintained and the amount of proteins in the obtained culture or its fraction can be further increased. In addition, variations caused by the animal serum in the serum-containing medium can be reduced. Furthermore, the culturing costs may be effectively decreased by about 50%. Table 1 shows components and their amounts of the 1:1 mixture of DMEM and Ham's F-12.

TABLE 1

| | | |
|---|---|---|
| | Concentration | Morality |
| Components | (mg/L) | (mM) |

Amino acid

| | | |
|---|---|---|
| D-Pantothenic Acis | 2.24 | 0.00895 |
| Glycine | 18.75 | 0.25 |
| L-Alanine | 4.45 | 0.05 |
| L-Arginine | 147.5 | 0.699 |
| hydrochloride | | |
| L-Asparagine-H₂O | 7.5 | 0.05 |
| L-Aspartic acid | 6.65 | 0.05 |
| L-Cysteine | 17.56 | 0.0998 |
| hydrochloride-H₂O | | |
| L-Cystine 2HCl | 31.29 | 0.1 |
| L-Glutamic Acid | 7.35 | 0.05 |
| L-Glutamine | 365 | 2.5 |
| L-Histidine | 31.48 | 0.15 |
| hydrochloride-H₂O | | |
| L-Isoleucine | 54.47 | 0.416 |
| L-Leucine | 59.05 | 0.451 |
| L-Lysine hydrochloride | 91.25 | 0.499 |
| L-Methionine | 17.24 | 0.116 |
| L-Phenylalanine | 35.48 | 0.215 |
| L-Proline | 17.25 | 0.15 |
| L-Serine | 26.25 | 0.25 |
| L-Threonine | 53.45 | 0.449 |
| L-Tryptophan | 9.02 | 0.0442 |
| L-Tyrosine disodium | 55.79 | 0.214 |
| salt dihydrate | | |
| L-Valine | 52.85 | 0.452 |

Vitamins

| | | |
|---|---|---|
| Biotin | 0.0035 | 0.0000143 |
| Choline chloride | 8.98 | 0.0641 |
| D-Calcium pantothenate | 2.24 | 0.0047 |
| Folic Acid | 2.65 | 0.00601 |
| i-Inositol | 12.6 | 0.07 |
| Niacinamide | 2.02 | 0.0166 |
| Pyridoxine hydrochloride | 2.031 | 0.00986 |
| Riboflavin | 0.219 | 0.000582 |
| Thiamine hydrochloride | 2.17 | 0.00644 |
| Vitamin B12 | 0.68 | 0.000502 |

Inorganic Salts

| | | |
|---|---|---|
| Calcium Chloride (CaCl₂) | 116.6 | 1.05 |
| (anhyd.) | | |
| Cupric sulfate | 0.0013 | 0.0000052 |
| (CuSO₄—5H₂O) | | |
| Ferric Nitrate | 0.05 | 0.000124 |
| (Fe(NO₃)₃″9H₂O) | | |
| Ferric sulfate (FeSO₄—7H₂O) | 0.417 | 0.0015 |
| Magnesium Chloride | 28.64 | 0.301 |
| (anhydrous) | | |
| Magnesium Sulfate (MgSO₄) | 48.84 | 0.407 |
| (anhyd.) | | |
| Potassium Chloride (KCl) | 311.8 | 4.16 |
| Sodium Chloride (NaCl) | 6995.5 | 120.61 |
| Sodium Phosphate dibasic | 71.02 | 0.5 |
| (Na₂HPO₄) anhydrous | | |
| Sodium Phosphate | 54.3 | 0.45257 |
| monobasic (NaH₂PO₄) | | |
| anhydrous | | |
| Zinc sulfate (ZnSO₄—7H₂O) | 0.432 | 0.0015 |

As described above, the pharmaceutical composition according to the present invention includes a culture of adult stem cells obtained from a culture solution or a fraction thereof. The culture of adult stem cells may be a supernatant obtained through centrifuging a culture solution of the adult stem cells; a concentrated product of the supernatant; or a freeze-dried product of the supernatant. The fraction of the culture may be a fraction obtained by fractionating a supernatant obtained through centrifuging a culture solution of the adult stem cells such that proteins having 36 to 700 kDa, preferably 36 to 45 kDa or 95 to 200 kDa, more preferably 36 to 45 kDa or 130 to 200 kDa, of molecular weight are contained in the resultant fraction; a concentrated product of the fraction; or a freeze-dried product of the fraction.

The centrifugation may be performed in a condition, where stem cells, macro molecules, and medium components can be removed by precipitation. For example, the centrifugation may be performed at 300-600×g and for 5 to 10 minutes, specifically at about 300×g for about 5 minutes. When required, the resultant product may be filtered with about 0.22 μm-syringe filter to remove the residual stem cells and unknown macro molecules. The concentrated product may be obtained by concentrating the culture or its fraction according to a conventional method, such as concentrating under reduced pressure, until the amount of proteins reaches to desired level.

In addition, the fractionation may be performed using a conventional membrane having a specific pore size. For example, a supernatant obtained by centrifuging a culture solution may be passed through a membrane having a pore size of about 1000 kDa, and then the resultant fractions may be passed through a membrane having appropriate pore sizes. The fractionation may be performed using liquid chromatography. For example, HW55F beads (TOSOH Co.) as a stationary phase is filled in a column having a diameter of 1 cm and a length of 60 cm, and a culture of the stem cells (if desired, a concentrate thereof) is added thereto. Then, phosphate buffered saline, as a mobile phase, is flowed through the column at an appropriate flow rate, e.g., at 0.5 ml/min, to obtain fractions in tubes. Optionally, the obtained fractions may be concentrated with a filter having an appropriate pore size or with conventional methods, e.g., concentration under reduced pressure, until desired concentration level is obtained.

The cancer to which the composition of the present invention is applicable includes various cancers, such as melanoma, pancreatic cancer, breast cancer, hepatic cancer, gastric cancer, colon cancer, lung cancer, or cervical cancer.

The pharmaceutical composition according to the present invention may further include a pharmaceutically acceptable carrier, in addition to a culture of adult stem cells or a fraction of the culture as an active ingredient. The pharmaceutical composition may be formulated into various parenteral dosage forms, such as liquids, suspensions, emulsions, lotions, ointments, or freeze-dried forms, according to a conventional method. Specifically, the pharmaceutical composition may be formulated into transdermal dosage forms, such as solutions for external use, emulsions, or ointments. Examples of the pharmaceutically acceptable carrier include an aqueous diluent or solvent, such as phosphate buffered saline, purified water, or fertilized water; and a non-aqueous diluent or solvent, such as propylene glycol, polyethylene glycol, or olive oil. In addition, when required, the pharmaceutical composition may further include a wetting agent, a fragrance, or a preservative.

The dosage of the culture of adult stem cells or its fraction included in the pharmaceutical composition may differ according to the patent's state and body weight, a degree of development of disease, dosage forms, routes and time period of administration, but may be appropriately determined by one of those skilled in the art. For example, the culture of adult stem cells or its fraction may be administered in an effective amount ranging 10 to 1000 ng/kg, preferably 50 to 500 ng/kg, and more preferably about 100 ng/kg, per day, and the administration may be performed once or several times a day. The pharmaceutical composition according to the present invention can be administered alone or together with other anti-cancer drugs, and in the latter case, the pharmaceutical composition according to the present and other drugs may be administered sequentially or at the same time. When the pharmaceutical composition according to the present is administered alone or together with other drugs, the pharmaceutical composition according to the present may be administered in as small amount as possible while the maximum effect can be secured without any side effects, and such an amount may be obvious to one of ordinary skill in the art.

The present invention will be described in further detail with reference to the following examples. These examples are for illustrative purposes only and are not intended to limit the scope of the present invention.

### EXAMPLE 1

### Isolation of Adipose Stem Cells

Adiopose tissues, which had been removed from a patient through liposuction and discarded, were collected from a patent after obtaining his/her approval. An extracellular matrix of the collected adipose tissues was treated with 0.075% collagenase at about 37° C. for 45 minutes in an about 5% CO₂ incubator, and then the obtained adipose tissues were centrifuged at about 1200×g for 5 minutes, thereby obtaining a stromal blood fraction including high-density stem cells. The obtained fraction was washed with PBS and then filtered with a 70 μm nylon cell filter to remove other tissues therefrom. Then, Histopaque-1077 (Sigma Co.) was used to separate mononuclear cells from a red blood-containing cell fragments.

The separated mononuclear cells were cultured in a Dulbecco's Modified Eagle's Medium (DMEM) supplemented with supplemented with 10% fetal bovine serum (FBS), 1% penicillin-streptomycin, at about 37° C., in about 5% CO₂ incubator, for 24 hours. And then, non-adhesive cells were removed to isolate adipose stem cells (about 1×10⁶ cells).

### EXAMPLE 2

### Cultivation of Stem Cell

4×10⁵ of adipose stem cells obtained in Example 1 were added to a DMEM supplemented with 1000 mg/L of D-glucose, 584 mg/L of L-glutamine, 110 mg/L of sodium pyruvate, 10% FBS, and 1% penicillin-streptomycin, and then sub-cultured in a 5% CO₂ incubator at about 90% of humidity at about 37° C. for 30 days.

The culture solution was removed from the sub-cultured culture using a pipette, and then the obtained cells were washed with a phosphate buffer three times. The obtained cells were inoculated at a concentration of 1.2×10⁶ cell/dish to a mixed medium of DMEM and Ham's F-12 in a weight ratio of 1:1, supplemented with 365 mg/L of L-glutamine, 15 mM of HEPES, and 55 mg/L of sodium pyruvate, and then cultured in a hypoxia condition for 72 hours.

### EXAMPLE 3

### Preparation of Concentrated Product of Stem Cell Culture and Fraction Thereof

500 ml of the culture solution finally obtained in Example 2 was centrifuged at 300×g for 5 minutes and precipitated stem cell pellets were removed. The obtained supernatant was filtered with a 0.22 μm-syringe filter to remove residual stem cells and unknown macro molecules. The obtained solution was divided into two aliquots. One aliquot was concentrated under reduced pressure to obtain 5 ml of a concentrated product. The obtained concentrated product was lyophilized and stored at −70° C.

The other aliquot, in which residual stem cells and unknown macro molecules are removed, was passed through a membrane having a pore size of 10 kDa (Amicon Ultra, Milli pore Co.) to remove peptide having less than 10 kDa of molecular weight and the culture medium. Proteins that were not passed through the membrane were obtained in a concentrated form. The concentrate was diluted with phosphate buffered saline (PBS), and the diluted solution was filtered using a 0.22 μm-syringe filter.

A column having a diameter of 1 cm and a length of 60 cm, which was filled with HW55F beads (TOSOH Co.), with a separation range of 10 to 700 kDa was connected to an AKTA FPLC system (Amersham biosciences Co.). The obtained concentrate in the above was fractionized by liquid chromatography using PBS as a mobile phase. The column was stabilized using 50 ml of PBS at a flow rate of 0.5 ml/min, and 100 μl of the concentrate was introduced into the column. Then, the separation degrees of proteins were detected at a UV wavelength of 280 nm by injecting about 47 ml of PBS to the column at a flow rate of 0.5 ml/min. 1 ml of the eluted solution was collected for 2 minutes per each tube and then fractionized. Each of the fractions was lyophilized and stored at −70° C.

### EXAMPLE 4

### Preparation of Concentrated Products of Umbilical Cord Blood Stem Cell Culture and Bone Marrow Stem Cell Culture and Fractions Thereof

Human umbilical mesenchymal stem cells (catalog No. 7530 Sciencell Research Laboratories, US) were inoculated into a mixed medium of Dulbecco's Modified Eagle's Medium (DMEM) and Ham's F-12 in a weight ratio of 1:1 supplemented with 365 mg/L of L-glutamine, 15 mM of HEPES, and 55 mg/L of sodium pyruvate in a concentration of 1.2×10⁶ cell/dish, and then cultured in a hypoxia condition for 72 hours. Concentrated products of the umbilical cord blood stem cells and fractions thereof were obtained in the same manner as in Example 3, and respectively stored at −70° C.

Human bone marrow stem cells (catalog No. MSC-001F, StemCell Technologies, US) were treated in the same manner as described above to obtain concentrated products of bone marrow stem cells and fractions thereof, and they are respectively stored at −70° C.

### EXPERIMENTAL EXAMPLE 1

### Evaluation of Growth-Inhibiting Activity Against Melanoma Cells

(1) Evaluation of Growth-Inhibiting Activity Against Rodent Melanoma Cell Line

B16 cell line (B16-F0, CRL-6322), which is a rodent melanoma cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

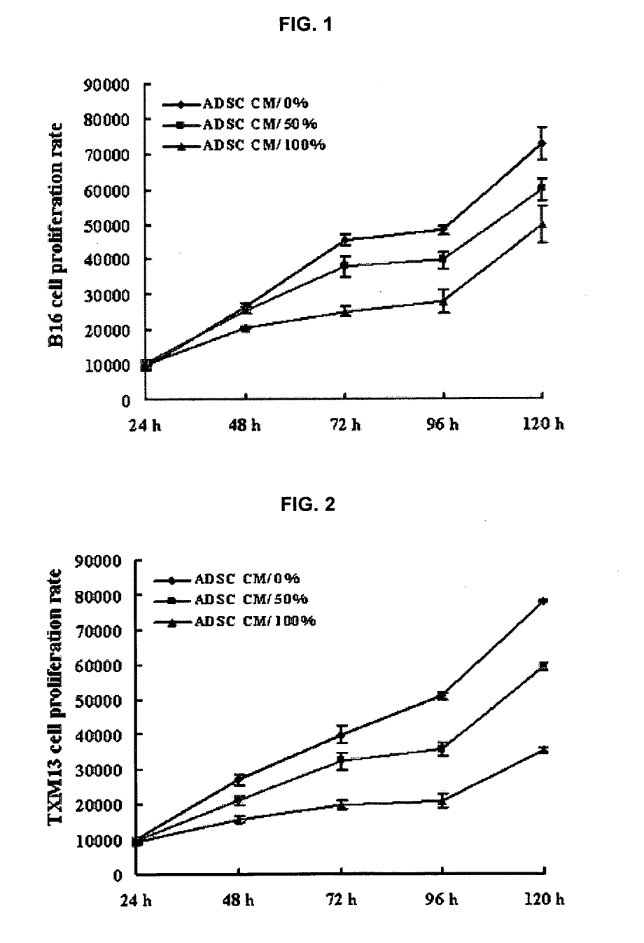

2×10³ of B16 cells were inoculated into each well of a 96-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/in of streptomycin), 50% of ADSC CM (that is, 100 μl of the mixed medium of DMEM and Ham's F-12 in a weight ratio of 1:1 supplemented with 10% FBS, 100 U/ml of penicillin, and 100 μg/ml of streptomycin +100 μl of ADSC CM), and 100% of ADSC CM (that is, 200 μl of ADSC CM). After 24, 48, 72, 96, and 120 hours, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 1.

Referring to FIG. 1, it can be seen that the growth of B16 cells was inhibited in proportion to the amount of the culture of adipose stem cells.

(2) Evaluation of Growth-Inhibiting Activity Against Human Melanoma Cell Line

TXM cell line (A7, CRL-2500), which is a human melanoma cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

2×10³ of TXM cells were inoculated into each well of a 96-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin), 50% of ADSC CM (that is, 100 μl of the mixed medium of DMEM and Ham's F-12 in a weight ratio of 1:1 supplemented with 10% FBS, 100 U/ml of penicillin, and 100 μg/ml of streptomycin +100 μl of ADSC CM), and 100% of ADSC CM (that is, 200 μl of ADSC CM). After 24, 48, 72, 96, and 120 hours, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 2.

Referring to FIG. 2, it can be seen that the growth of TXM cells was inhibited in proportion to the amount of the culture of adipose stem cells.

### EXPERIMENTAL EXAMPLE 2

### Evaluation of Growth-Inhibiting Activity Against Pancreatic Cancer Cells

Hpac cell line (PANC-1, CRL-1469), which is a human pancreatic cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

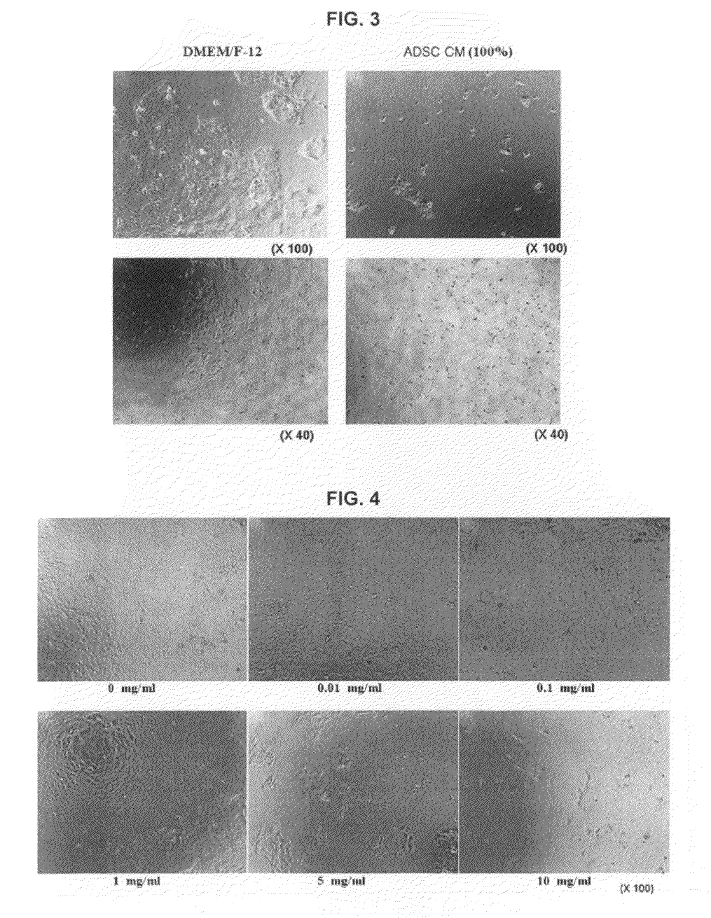

2×10³ of Hpac cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin), and 100% of ADSC CM (that is, 200 μl of ADSC CM). After 72 hours of the treatment, the images obtained by using a microscope (X40 and X100) are shown in FIG. 3. Referring to FIG. 3, it can be seen that the growth of Hpac cells was inhibited in the group treated with the culture of adipose stem cells.

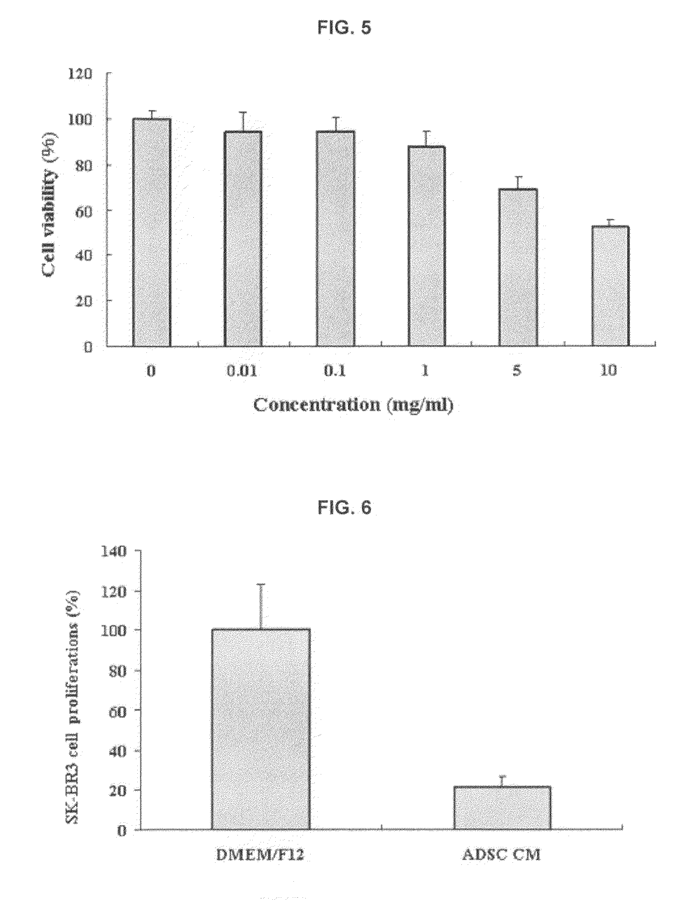

And also, 2×10³ of Hpac cells were inoculated into each well of a 24-well plate, and then treated with the concentrated product of adipose stem cells obtained in Example 3 (ADSC CM), each having 0, 0.01, 0.1, 1, 5, or 10 mg/ml of ADSC CM in 200 μl of the mixed medium of DMEM and Ham's F-12 in a weight ratio of 1:1 supplemented with 10% FBS, 100 U/ml of penicillin, and 100 μg/ml of streptomycin. After 72 hours of the treatment, the images obtained by using a microscope (X100) are shown in FIG. 3. And also, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 5. Referring to FIG. 4 and FIG. 5, it can be seen that the growth of Hpac cells was inhibited in proportion to the amount of the culture of adipose stem cells.

### EXPERIMENTAL EXAMPLE 3

### Evaluation of Growth-Inhibiting Activity Against Human Breast Cancer Cells

SK-BR3 cell line (MCF 10F, CRL-10318), which is a human breast cancer cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

4×10³ of SK-BR3 cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of ADSC CM (that is, 200 μl of ADSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 6.

Referring to FIG. 6, it can be seen that more than about 70% of the growth of SK-BR3 cells were inhibited in the group treated with the culture of adipose stem cells.

### EXPERIMENTAL EXAMPLE 4

### Evaluation of Growth-Inhibiting Activity Against Human Hepatic Cancer Cells

Hur7 cell line (SK-HEP-1, HTP-52), which is a human hepatic cancer cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

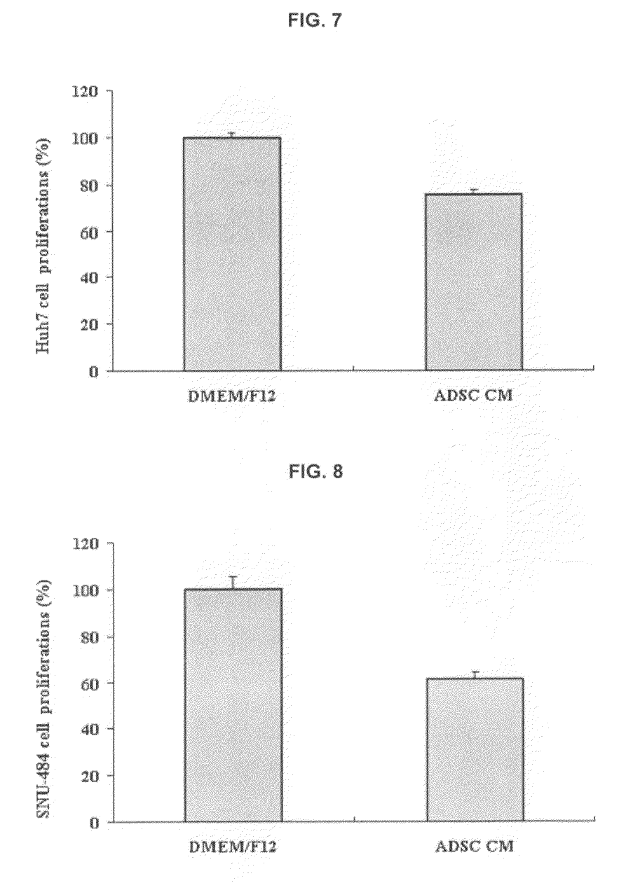

4×10³ of Hur7 cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of ADSC CM (that is, 200 μl of ADSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 7.

Referring to FIG. 7, it can be seen that more than about 20% of the growth of Hur7 cells were inhibited in the group treated with the culture of adipose stem cells.

### EXPERIMENTAL EXAMPLE 5

### Evaluation of Growth-Inhibiting Activity Against Human Gastric Cancer Cells

SNU-484 cell line (SNU-16, CRL-5974), which is a human gastric cancer cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

4×10³ of SNU-484 cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of ADSC CM (that is, 200 μl of ADSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 8.

Referring to FIG. 8, it can be seen that more than about 30% of the growth of SNU-484 cells were inhibited in the group treated with the culture of adipose stem cells.

### EXPERIMENTAL EXAMPLE 6

### Evaluation of Growth-Inhibiting Activity Against Human Colon Cancer Cells

RKO cell line (CRL-2577), which is a human colon cancer cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

4×10³ of RKO cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of ADSC CM (that is, 200 μl of ADSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 9.

Referring to FIG. 9, it can be seen that more than about 50% of the growth of RKO cells were inhibited in the group treated with the culture of adipose stem cells.

### EXPERIMENTAL EXAMPLE 7

### Evaluation of Growth-Inhibiting Activity Against Human Lung Cancer Cells

A549 cell line (CRL-185), which is a human hepatic cancer cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

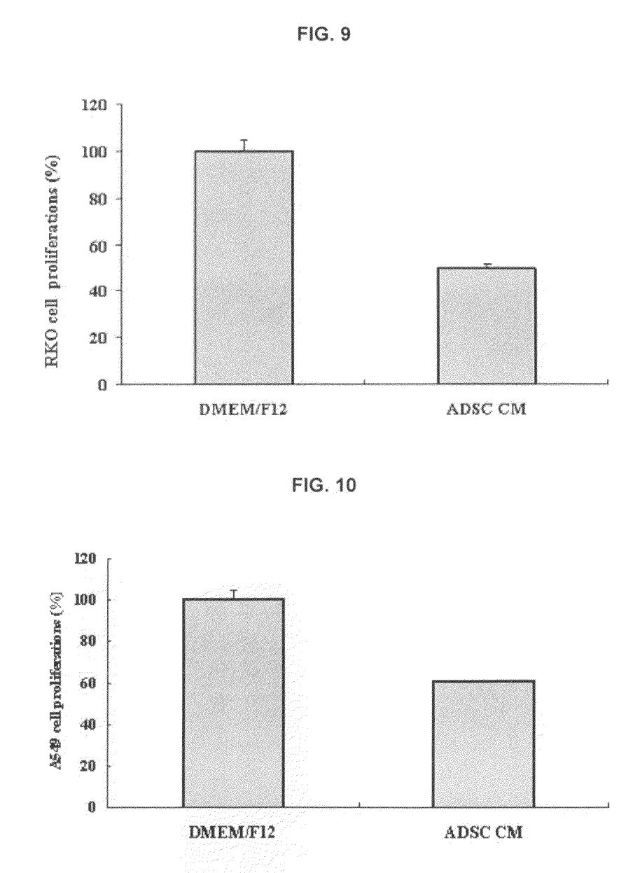

4×10³ of A549 cells were inoculated into each well of a 24-well plate, and then treated with the concentrated product of adipose stem cells obtained in Example 3 (ADSC CM), each being 0% of ADSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of ADSC CM (that is, 200 μl of ADSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 10.

Referring to FIG. 10, it can be seen that more than about 40% of the growth of A549 cells were inhibited in the group treated with the culture of adipose stem cells.

### EXPERIMENTAL EXAMPLE 8

### In Vivo Inhibition Test of Melanoma Growth

B16 cell line (B16-F0, CRL-6322), which is a rodent melanoma cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

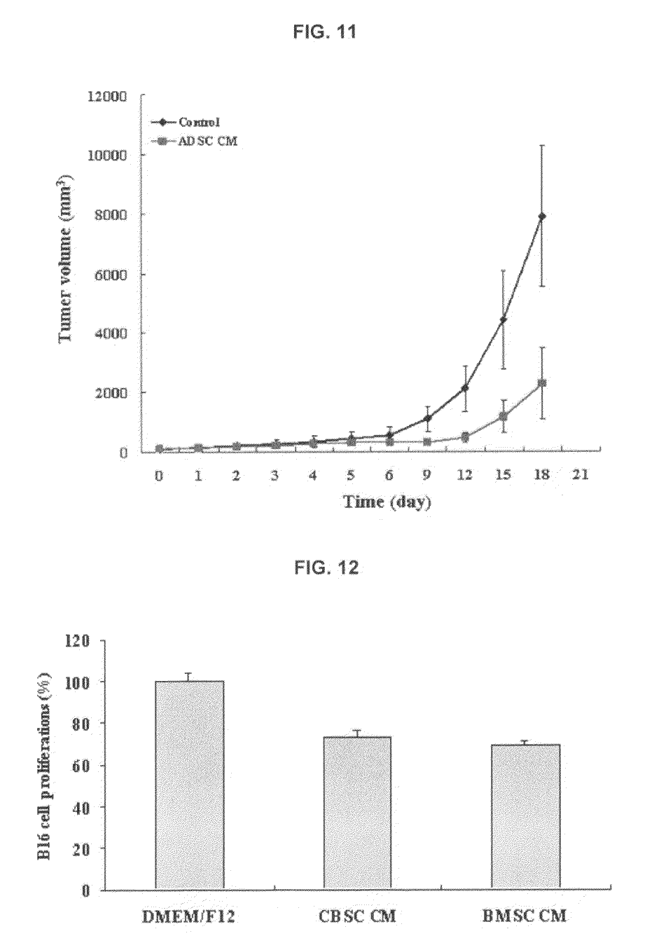

2×10⁶ of the B16 cells were subcutaneously injected into six-week-old B57BL/6 mice (n=12) to induce melanoma, and 6 mice were used as a control group, and the remaining 6 mice were used as a test group. When the volume of the tumor reached 50 to 160 mm³ after 3 to 5 days of the melanoma induction, 100 μl of the solution, which was obtained by diluting the concentrated product of adipose stem cell culture prepared in Example 3 in PBS to a concentration of 12.5 mg/ml, was injected into the tumors of the test group 6 times every other day. For the control group, 100 μl of PBS was injected 6 times into the tumors every other day. The size of the tumors of the mice of the control and test groups was measured by measuring the longest side and the shortest side with a vernier caliper, and the volume of the tumors were calculated. The results are shown in FIG. 11.

Referring to FIG. 11, it can be seen that the growth of tumors in the test group was significantly inhibited.

### EXPERIMENTAL EXAMPLE 9

### Evaluation of Growth-Inhibiting Activity Against Melanoma Cells

B16 cell line (B16-F0, CRL-6322), which is a rodent melanoma cell line, was cultured in a mixed medium of DMEM and Ham's F-12 in a weight ratio of 1:1 supplemented with 10% FBS, 100 U/ml of penicillin, and 100 μg/ml of streptomycin at 37° C., in 5% CO₂ incubator.

2×10³ of B16 cells were inoculated into each well of a 96-well plate, and then treated with the concentrated product of umbilical cord blood stern cells (CBSC CM) and the concentrated product of bone marrow stem cells (BMSC CM) obtained in Example 4, each being 0% of CBSC CM and BMSC CM (that is, 200 μl of the mixed medium of DMEM and Ham's F-12 in a weight ratio of 1:1 supplemented with 10% FBS, 100 U/ml of penicillin, and 100 μg/ml of streptomycin) and 100% of CBSC CM or BMSC CM (that is, 200 μl of CBSC CM or BMSC CM). After 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 12.

Referring to FIG. 12, it can be seen that the growths of B16 cells in the test groups were significantly inhibited.

### EXPERIMENTAL EXAMPLE 10

### Evaluation of Growth-Inhibiting Activity Against Melanoma Cells

B16 cell line (B16-F0, CRL-6322), which is a rodent melanoma cell line, was cultured in a DMEM supplemented with 10% FBS, 100 U/ml of penicillin, and 100 μg/ml of streptomycin at 37° C., in 5% CO₂ incubator.

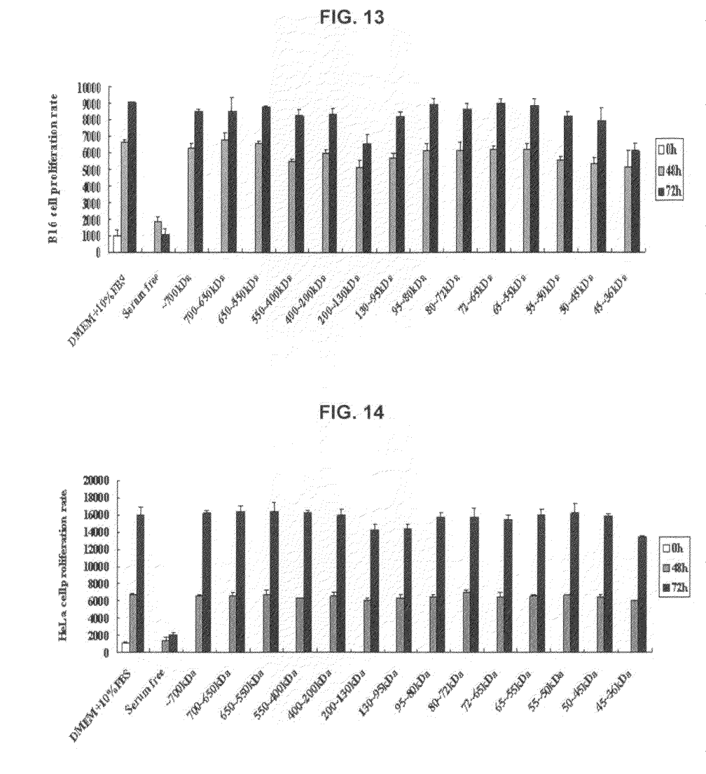

100 μl of DMEM supplemented with 10% FBS, 100 U/ml penicillin, and 100 μg/ml streptomycin was added to a 96-well plate, and 2×10³ of B16 cells were inoculated to each of the wells. Then, the B16 cells in the each well were treated with 2 ug of the lyophilized fraction of adipose stem cell culture prepared in Example 3. After 48 and 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 13.

Referring to FIG. 13, it can be seen that the growth of the B16 cells was respectively inhibited by 30% and 40% when treated with the fractions of the adipose stem cell culture solution having 130 to 200 kDa of molecular weight and 36 to 45 kDa of molecular weight.

### EXPERIMENTAL EXAMPLE 11

### Evaluation of Growth-Inhibiting Activity Against Cervical Cancer Cells

HeLa cell line (CCL-2), which is a rodent cervical cancer cell line, was cultured in a DMEM supplemented with 10% FBS, 100 U/ml of penicillin, and 100 μg/ml of streptomycin at 37° C., in 5% CO₂ incubator.

100 μl of DMEM supplemented with 10% FBS, 100 U/ml penicillin, and 100 μg/ml streptomycin was added to a 96-well plate, and 2×10³ of HeLa cells were inoculated to each of the wells. Then, the HeLa cells in the each well were treated with 2 ug of the lyophilized fraction of adipose stem cell culture prepared in Example 3. After 48 and 72 hours of the treatment, 10 μl of CCK-8 solution (Dojindo, Gaithersburg, Md.) was added to each well, and then cultured for 3 hours. Then, absorbance was measured at 450 nm using a microplate reader (TECAN, Gr, Austria). Cell proliferation rates corresponding to the absorbance were calculated using a standard curve, and the results are shown in FIG. 14.

Referring to FIG. 14, it can be seen that the growth of the HeLa cells was respectively inhibited by 20% and 25% when treated with the fractions of the adipose stem cell culture solution having 95 to 200 kDa of molecular weight and 36 to 45 kDa of molecular weight.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A pharmaceutical composition for treating cancer, comprising a culture of adult stem cells or a fraction of the culture as an active ingredient.

2. The pharmaceutical composition of claim 1, wherein the culture of adult stem cells is a supernatant obtained through centrifuging a culture solution of the adult stem cells; a concentrated product of the supernatant; or a lyophilized product of the supernatant.

3. The pharmaceutical composition of claim 1, wherein the fraction of the adult stem cell culture is a fraction obtained by fractionating a supernatant obtained through centrifuging a culture solution of the adult stem cells such that proteins having 36 to 700 kDa of molecular weight are contained in the resultant fraction; a concentrated product of the fraction; or a lyophilized product of the fraction.

4. The pharmaceutical composition of claim 1, wherein the fraction of the adult stem cells is a fraction obtained by fractionating a supernatant obtained through centrifuging a culture solution of the adult stem cells such that proteins having 36 to 45 kDa or 95 to 200 kDa of molecular weight are contained in the resultant fraction; a concentrated product of the fraction; or a lyophilized product of the fraction.

5. The pharmaceutical composition of claim 1, wherein the fraction of the adult stem cells is a fraction obtained by fractionating a supernatant obtained through centrifuging a culture solution of the adult stem cells such that proteins having 36 to 45 kDa or 130 to 200 kDa of molecular weight are contained in the resultant fraction; a concentrated product of the fraction; or a lyophilized product of the fraction.

6. The pharmaceutical composition of claim 2, wherein the culture solution of the adult stem cells is obtained by sub-culturing the adult stem cells in a serum-containing medium, followed by culturing the sub-cultured adult stem cells in a serum-free medium.

7. The pharmaceutical composition of claim 6, wherein the serum-containing medium is a Dulbecco's Modified Eagle's Medium (DMEM) supplemented with 1 to 2 mM of glutamine, 0.5 to 1 mM of sodium pyruvate, 0.1 to 10% of fetal bovine serum (FBS), 1% of antibiotics (100 lU/ml), and 1 to 4.5 g/L glucose.

8. The pharmaceutical composition of claim 6, wherein the serum-free medium is a mixed medium of DMEM and Ham's F-12.

9. The pharmaceutical composition of claim 1, wherein the adult stem cells are derived from fat, bone marrow, umbilical cord blood, or placenta.

10. The pharmaceutical composition of claim 1, wherein the cancer is melanoma, pancreatic cancer, breast cancer, hepatic cancer, gastric cancer, colon cancer, lung cancer, or cervical cancer.

11. The pharmaceutical composition of claim 3, wherein the culture solution of the adult stem cells is obtained by sub-culturing the adult stem cells in a serum-containing medium, followed by culturing the sub-cultured adult stem cells in a serum-free medium.

12. The pharmaceutical composition of claim 4, wherein the culture solution of the adult stem cells is obtained by sub-culturing the adult stem cells in a serum-containing medium, followed by culturing the sub-cultured adult stem cells in a serum-free medium.

13. The pharmaceutical composition of claim 5, wherein the culture solution of the adult stem cells is obtained by sub-culturing the adult stem cells in a serum-containing medium, followed by culturing the sub-cultured adult stem cells in a serum-free medium.

14. The pharmaceutical composition of claim 2, wherein the adult stem cells are derived from fat, bone marrow, umbilical cord blood, or placenta.

15. The pharmaceutical composition of claim 3, wherein the adult stem cells are derived from fat, bone marrow, umbilical cord blood, or placenta.

16. The pharmaceutical composition of claim 4, wherein the adult stem cells are derived from fat, bone marrow, umbilical cord blood, or placenta.

17. The pharmaceutical composition of claim 5, wherein the adult stem cells are derived from fat, bone marrow, umbilical cord blood, or placenta.

18. The pharmaceutical composition of claim 2, wherein the cancer is melanoma, pancreatic cancer, breast cancer, hepatic cancer, gastric cancer, colon cancer, lung cancer, or cervical cancer.

19. The pharmaceutical composition of claim 3, wherein the cancer is melanoma, pancreatic cancer, breast cancer, hepatic cancer, gastric cancer, colon cancer, lung cancer, or cervical cancer.

20. The pharmaceutical composition of claim 4, wherein the cancer is melanoma, pancreatic cancer, breast cancer, hepatic cancer, gastric cancer, colon cancer, lung cancer, or cervical cancer.
